(19) United States
(12) Patent Application Publication
(10) Pub. No.: US 2020/0170774 A1
(43) Pub. Date: Jun. 4, 2020

(54) IN-VIVO INDWELLING TUBE AND METHOD FOR PRODUCING SAME

(71) Applicant: KANEKA CORPORATION, Osaka-shi, Osaka (JP)

(72) Inventors: Tomoharu KOISO, Settsu-shi (JP); Jiaying LIU, Settsu-shi (JP); Youhei KUROSE, Settsu-shi (JP)

(73) Assignee: KANEKA CORPORATION, Osaka-shi, Osaka (JP)

(21) Appl. No.: 16/616,351

(22) PCT Filed: Jun. 7, 2018
(86) PCT No.: PCT/JP2018/021863

§ 371 Date: Nov. 22, 2019

(30) Foreign Application Priority Data
Jun. 13, 2017 (JP) 2017-115569

(51) Int. Cl. A61F 2/04; A61F 2/95

(52) U.S. Cl. CPC A61F 2/04; A61F 2002/041; A61F 2240/001; A61F 2/95

(57) ABSTRACT

Provided is an in-vivo indwelling tube having a high flap strength and capable of smoothing the passing through a conduit of an endoscope. A method for producing an in-vivo indwelling tube having one end and the other end, the method including the steps of: arranging, in a lumen of the first tubular member, the other end of the second tubular member; joining the first tubular member and the second tubular member; arranging the second tubular member in a lumen of a third tubular member in which an axial length is shorter than the second tubular member; joining the second tubular member and the third tubular member; arranging one end of the second tubular member in a lumen on the other end side of the fourth tubular member including a flap with a base end and a free end; and joining the second tubular member and the fourth tubular member.

### TECHNICAL FIELD

The present invention relates to a tube to be placed in a living body to prevent occlusion or constriction of a lumen in the living body and a method for producing same.

### BACKGROUND ART

An in-vivo indwelling tube represented by stents, particularly stents for bile ducts or pancreatic ducts are medical devices for treating various diseases such as biliary obstruction, jaundice, biliary tract cancer, and the like caused by constriction or occlusion of a lumen in the living body such as bile duct, pancreatic duct, and the like. The in-vivo indwelling tube is indwelled in the lumen in the living body for the purpose of draining bile from the bile duct to the duodenum and maintaining the inner diameter of the lumen by expanding the lesion at the constricted or occluded site from the inner side. When the tissue of a lesion such as a cancer cell enters the lumen of the in-vivo indwelling tube and the lumen of the in-vivo indwelling tube is occluded or constricted, the in-vivo indwelling tube needs to be replaced.

The in-vivo indwelling tube includes one made of a metal material and one made of a resin material. In the treatment as described above, an in-vivo indwelling tube made of a resin material may be used.

First, a conventional in-vivo indwelling tube will be described with reference to FIG. 16. As shown in FIG. 16, an in-vivo indwelling tube 201 made of a resin material includes a proximal end 202 and a distal end 203 and extends in a perspective direction. Generally, the in-vivo indwelling tube 201 has a cut on a proximal outer surface to form a proximal flap 205 and has a cut on a distal outer surface to form a distal flap 208 (e.g., Patent Documents 1 to 3). The proximal flap 205 and the distal flap 208 have the function of securing the in-vivo indwelling tube 201 in the lumen in the living body. When the in-vivo indwelling tube 201 is a bile duct stent, for example, the distal flap 208 is placed on the distal side than the constricted portion (occluded portion) of the bile duct so that the in-vivo indwelling tube does not fall off from the bile duct toward the duodenum side, and the proximal flap 205 is placed near the papilla of the duodenum so that the proximal end 202 of the in-vivo indwelling tube 201 does not penetrate into the bile duct.

Normally, the proximal flap 205 extends from the proximal side to the distal side of the in-vivo indwelling tube 201 and toward the outside in the radial direction. Furthermore, the proximal flap 205 is formed to open toward the outside in the radial direction to prevent the proximal end 202 of the in-vivo indwelling tube 201 from entering the bile duct. When such an in-vivo indwelling tube 201 is inserted into the conduit of an endoscope, the inner wall of the conduit and the proximal flap 205 come into contact and the proximal flap 205 bends, making it difficult for the tube to pass through and arising a problem that the delivery of the in-vivo indwelling tube 201 to a desired indwelling site is difficult. In order to prevent the bending of the proximal flap 205, an introduction member is known which reduces the conduit diameter of a portion through which the in-vivo indwelling tube is inserted by closing a part of the branched part of the conduit in the endoscope (e.g., Patent Document 4).

### PRIOR ART DOCUMENTS

### Patent Documents

Patent Document 1: JP-A-2015-36043

Patent Document 2: JP-A-9-56809

Patent Document 3: JP-A-5-192389

Patent Document 4: JP-A-2006-87712

### SUMMARY OF THE INVENTION

### Problems to be Solved by the Invention

In the in-vivo indwelling tube disclosed in Patent Document 1, the outer periphery of a tubular member is axially cut to form a flap. As a result, a hole having a size of the same extent as the flap and communicating the exterior of the tubular member and the lumen exists at the lower part of the flap of the tubular member. For example, when there is a lesion of cancer near the papilla of the duodenum, the vicinity of the proximal flap of the in-vivo indwelling tube may come into contact with the lesion. If a hole having a size that allows the lesion to enter is formed in the vicinity of the proximal flap, cancer cells may enter the lumen of the in-vivo indwelling tube through the hole, thus occluding or constricting the lumen of the in-vivo indwelling tube.

In the in-vivo tube disclosed in Patent Document 2, the side wall of the tubular member is cut shallow to form a proximal flap. Such an in-vivo tube has problems in that the strength of the proximal flap is low and the proximal end of the in-vivo indwelling tube cannot be sufficiently prevented from entering the bile duct or the like, and in that the proximal flap is easily broken.

In the in-vivo indwelling tube disclosed in Patent Document 3, a reinforcement blade is placed between the outer tube and the inner tube. This blade increases the strength of the entire in-vivo indwelling tube. Therefore, such an in-vivo indwelling tube has problems in that it is difficult to pass the in-vivo indwelling tube through the conduit of the endoscope and that it is difficult to deliver the in-vivo indwelling tube to a desired indwelling site.

Furthermore, since the introduction member as in Patent Document 4 has a configuration in which the outer diameter is larger than the inner diameter of the conduit of the general endoscope, the introduction member cannot be inserted into the conduit of the general endoscope and thus has low versatility and is difficult to use.

In view of the situations described above, it is an object of the present invention to provide an in-vivo indwelling tube having a high flap strength and capable of smoothing the passing through a conduit of an endoscope or the like, and a method for producing the same.

### Solutions to the Problems

A method for manufacturing an in-vivo indwelling tube of the present invention that has solved the above problems comprising is a method for producing an in-vivo indwelling tube having one end and the other end comprising: a first process of arranging the other end of a second tubular member in a lumen of the first tubular member; a second process of joining the first tubular member and the second tubular member; a third process of arranging the second tubular member in a lumen of a third tubular member that its length in an axial direction is shorter than the second tubular member; a fourth process of joining the second tubular member and the third tubular member; a fifth process of arranging one end of the second tubular member in a lumen on the other end side of the fourth tubular member including a flap having a base end and a free end; and a sixth process of joining the second tubular member and the fourth tubular member.

The method for manufacturing an in-vivo indwelling tube is preferable wherein a thickness of the second tubular member is thinner than thicknesses of the first tubular member, the third tubular member, and the fourth tubular member.

The method for manufacturing an in-vivo indwelling tube is preferable wherein a thickness of the flap of the fourth tubular member is thicker than the thickness of at least either the first tubular member or the third tubular member.

The method for manufacturing an in-vivo indwelling tube is preferable wherein a thickness of the flap of the fourth tubular member is thinner than the thickness of at least either the first tubular member or the third tubular member.

The method for manufacturing an in-vivo indwelling tube is preferable wherein a type A durometer hardness of a material that constitutes the fourth tubular member is higher than a type A durometer hardness of a material that constitutes the first tubular member, the second tubular member, and the third tubular member.

The method for manufacturing an in-vivo indwelling tube is preferable wherein an inner diameter of one end of the first tubular member is larger than an inner diameter of the other end of the first tubular member.

The method for manufacturing an in-vivo indwelling tube is preferable wherein in the fourth process, the first tubular member and the third tubular member are joined.

The method for manufacturing an in-vivo indwelling tube is preferable wherein in the sixth process, the third tubular member and the fourth tubular member are joined.

The method for manufacturing an in-vivo indwelling tube is preferable wherein before the sixth process, one end of the third tubular member is arranged on one end side or the other end side of the fourth tubular member of the base end of the flap of the fourth tubular member.

The method for manufacturing an in-vivo indwelling tube is preferable wherein the second process and the fourth process, or the second process, the fourth process, and the sixth process are performed by a simultaneous heating process.

The method for manufacturing an in-vivo indwelling tube is preferable wherein further comprising, before the first process, a process of arranging a core material in a lumen of the second tubular member.

The method for manufacturing an in-vivo indwelling tube is preferable wherein further comprising, after the sixth process, a process of arranging a supporting member radially outward of at least either one end side of the base end of the flap of the fourth tubular member, or the other end side of the base end of the flap of the fourth tubular member and one end side of a midpoint of the first tubular member.

The in-vivo indwelling tube is preferable wherein comprising: a tubular member having a proximal side and a distal side; a proximal flap, having a base end on a proximal side and a free end on a distal side, on the proximal side of the tubular member; and a distal flap, having a base end on a distal side and a free end on a proximal side, on the distal side of the tubular member, wherein the tubular member includes a larger diameter portion having a maximum outer diameter larger than an average outer diameter of the tubular member between a position of the tubular member corresponding to the free end of the distal flap and a position of the tubular member corresponding to the free end of the proximal flap, and the larger diameter portion is on at least either the proximal side of the base end of the proximal flap or the distal side of the base end of the distal flap.

The in-vivo indwelling tube is preferable wherein comprising: a tubular member having a proximal side and a distal side; a proximal flap, having a base end on a proximal side and a free end on a distal side, on the proximal side of the tubular member; and a distal flap, having a base end on a distal side and a free end on a proximal side, on the distal side of the tubular member, wherein the tubular member includes a smaller diameter portion having a minimum outer diameter smaller than an average outer diameter of the tubular member between a position of the tubular member corresponding to the free end of the distal flap and a position of the tubular member corresponding to the free end of the proximal flap, and the smaller diameter portion is on at least either between the base end of the proximal flap and the position on the proximal side of the free end of the proximal flap when the proximal flap is in a closed state or between the base end of the distal flap and a position on the distal side of the free end of the distal flap when the distal flap is in a closed state.

The in-vivo indwelling tube is preferable wherein the tubular member has a hole in the smaller diameter portion.

The in-vivo indwelling tube is preferable wherein the tubular member includes, radially outward of the tubular member, a supporting member on at least one of distal side of a midpoint of the base end and the free end of the distal flap, a proximal side of the free end of the distal flap and the distal side of the midpoint of the tubular member, a proximal side of a midpoint of the base end and the free end of the proximal flap, and a distal side of the free end of the proximal flap and the proximal side of the midpoint of the tubular member.

The in-vivo indwelling tube is preferable wherein the tubular member includes a first region and a second region sequentially from the proximal side of the tubular member, and colors of the first region and the second region differ from each other on the distal side of the base end of the proximal flap.

The in-vivo indwelling tube is preferable wherein a thickness of the distal flap or the proximal flap is thinner than a thickness of a proximal end of the tubular member.

The in-vivo indwelling tube is preferable wherein the thickness of the proximal flap is an average thickness or more of the tubular member between the position of the tubular member corresponding to the free end of the proximal flap and the position of the tubular member corresponding to the free end of the distal flap.

The in-vivo indwelling tube is preferable wherein a type A durometer hardness of a material that constitutes the proximal flap or the distal flap is higher than an average type A durometer hardness of a material that constitutes the tubular member between the position of the tubular member corresponding to the free end of the distal flap and the position of the tubular member corresponding to the free end of the proximal flap.

### Effects of the Invention

The in-vivo indwelling tube itself can be made flexible but the flap strength can be increased by producing the in-vivo indwelling tube through the production method according to the present invention.

### BRIEF DESCRIPTION OF THE DRAWINGS

FIG. 1 shows a side view of a delivery system of an in-vivo indwelling tube according to an embodiment of the present invention.

FIG. 2 is a process cross-sectional view of a first process in the embodiment of the present invention.

FIG. 3 is a process cross-sectional view of a second process in the embodiment of the present invention.

FIG. 4 is a process cross-sectional view of a third process in the embodiment of the present invention.

FIG. 5 is a process cross-sectional view of a fourth process in the embodiment of the present invention.

FIG. 6 is a process cross-sectional view of a fifth process in the embodiment of the present invention.

FIG. 7 is a process cross-sectional view of a sixth process in the embodiment of the present invention.

FIG. 8 is a process cross-sectional view of an example of a state before the sixth process in the embodiment of the present invention.

FIG. 9 is a process cross-sectional view of another example of the state before the sixth process in the embodiment of the present invention.

FIG. 10 is a process cross-sectional view of an example of a state after the sixth process in the embodiment of the present invention.

FIG. 11 shows a side view of the in-vivo indwelling tube according to the embodiment of the present invention.

FIG. 12 shows a side view of an example of the in-vivo indwelling tube according to the embodiment of the present invention.

FIG. 13 shows a side view of another example of the in-vivo indwelling tube according to the embodiment of the present invention.

FIG. 14 shows a side view when a proximal flap is in a closed state of another example of the in-vivo indwelling tube according to the embodiment of the present invention.

FIG. 15 shows a side view of a still another example of the in-vivo indwelling tube according to the embodiment of the present invention.

FIG. 16 shows a side view of a conventional in-vivo indwelling tube.

FIG. 17 shows an enlarged side view in the vicinity of the proximal flap when the proximal flap is in the closed state of the conventional in-vivo indwelling tube.

### MODE FOR CARRYING OUT THE INVENTION

Hereinafter, the present invention will be more specifically described based on the following embodiments, but the present invention is not limited by the following embodiments as a matter of course, and modifications can be appropriately made within a scope which can conform to the description made above and below, all of which being encompassed with in the technical scope of the present invention. In each drawing, hatching, member reference symbols, and the like are sometimes omitted for the sake of convenience, but in such a case, specification and other drawings are to be referred to. Furthermore, the dimensions of various members in the drawings may differ from the actual dimensions, as priority is given to helping to understand the features of the present invention.

The in-vivo indwelling tube is used by being attached to a delivery system (conveying device) such as a catheter having a site to install the in-vivo indwelling tube in order to convey the in-vivo indwelling tube to the lesion.

In the present invention, the proximal side refers to the direction on the hand side of the user (operator) with respect to the extending direction of the in-vivo indwelling tube, and the distal side refers to the direction opposite to the proximal side (that is, direction on treatment target side). In addition, the direction from the proximal side to the distal side of the in-vivo indwelling tube is referred to as an axial direction. The radial direction refers to the radius direction of the tubular member, inside in the radial direction refers to the direction toward the axial center side of the tubular member, and outside in the radial direction refers to the radial direction toward the side opposite side to the inner side.

In the following embodiment, a case where one end is a proximal end, that is, the production processes of the flap on the proximal side will be described, but the present embodiment can also be applied to the production of the flap on the distal side. In such a case, the description of the embodiment should be understood by reversing the description of perspective direction. The embodiment may be applied to both flaps, or may be applied to only one flap.

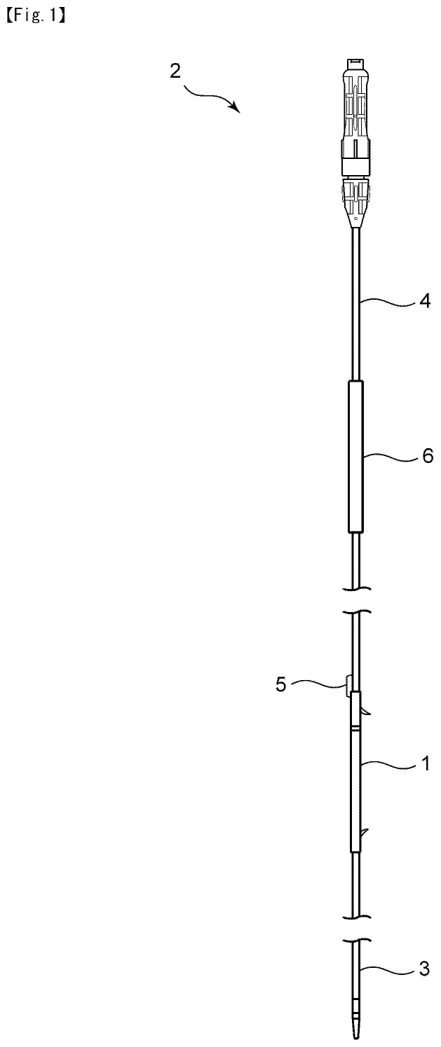

Before describing the method for producing an in-vivo indwelling tube of the present invention in detail, a configuration example of a delivery system for delivering an in-vivo indwelling tube to an indwelling target site will be first described with reference to FIG. 1. An example of the delivery system is shown in FIG. 1. In the delivery system 2, an outer catheter 4 and the in-vivo indwelling tube 1 are placed on the outer side in the radial direction of the inner catheter 3. The in-vivo indwelling tube 1 and the outer catheter 4 are coupled by a suture thread 5. As the in-vivo indwelling tube 1 and the outer catheter 4 are coupled to each other, when being conveying to the lesion, the in-vivo indwelling tube 1 can be pulled back in the lumen in the living body to finely adjust the position. Therefore, the in-vivo indwelling tube 1 can be easily indwelled at an appropriate position of the lesion. The insertion auxiliary tube 6 is placed on the outer side in the radial direction of the outer catheter 4. The insertion auxiliary tube 6 can make it difficult for the flap to be folded back during conveyance of the in-vivo indwelling tube 1, and can prevent kinking of the delivery system 2 at the time of insertion. As a result, the in-vivo indwelling tube 1 can be conveyed smoothly.

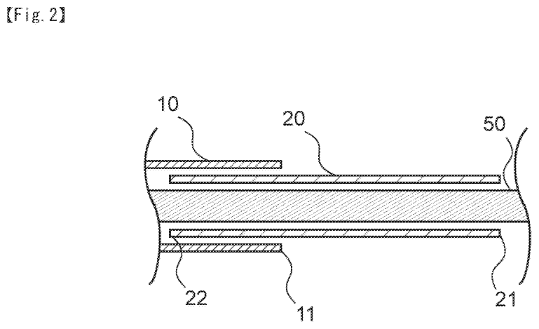

In the present invention, a method for producing an in-vivo indwelling tube having one end and the other end includes a first process of arranging, in a lumen of a first tubular member, the other end of a second tubular member, a second process of joining the first tubular member and the second tubular member, a third process of arranging the second tubular member in a lumen of a third tubular member that its length in an axial direction is shorter than the second tubular member, a fourth process of joining the second tubular member and the third tubular member, a fifth process of arranging one end of the second tubular member in a lumen on the other end side of the fourth tubular member including a flap having a base end and a free end, and a sixth process of joining the second tubular member and the fourth tubular member. Hereinafter, the method for producing the in-vivo indwelling tube according to the present invention will be described using FIGS. 2 to 7. In the following description, one end indicates the proximal end and the other end indicates the distal end, but the distal end may be the one end and the proximal end may be the other end. In FIGS. 2 to 7, the left direction in the plane of drawing corresponds to the distal side of the in-vivo indwelling tube, and the right direction in the plane of drawing corresponds to the proximal side of the in-vivo indwelling tube.

The length in the long axis direction of the in-vivo indwelling tube produced through the above processes and the outer diameter of the cross-section perpendicular to the long axis can be appropriately set according to the size of the lesion and the application site. Generally, the length of the in-vivo indwelling tube in the long axis direction is 200 mm or more and 700 mm or less, and the outer diameter of the cross-section perpendicular to the long axis is preferably 1.5 mm or more and 4.2 mm or less. The thickness of the in-vivo indwelling tube is preferably 0.2 mm or more and 0.6 mm or less. The shape of the cross-section perpendicular to the long axis of the in-vivo indwelling tube can be appropriately selected according to the lesion. In order to avoid damaging the body cavity, the shape of the cross-section can be circular, elliptical or the like.

A first tubular member 10, a second tubular member 20, a third tubular member 30, and a fourth tubular member 40 are tubular members extending in the axial direction, and can use, for example, resin tube molded by extrusion molding. The first tubular member 10, the third tubular member 30, and the fourth tubular member 40 are mainly placed on the outer side in the radial direction of the in-vivo indwelling tube. The second tubular member 20 is mainly placed on the inner side in the radial direction of the in-vivo indwelling tube.

Examples of a resin that constitutes each tubular member 10, 20, 30, 40 and the flap 43 include polyamide resins, polyester resins, polyurethane resins, polyolefin resins, fluorine resins, vinyl chloride resins, silicone resins, natural rubber, and the like. Only one kind of resin may be used or two or more kinds of resins may be simultaneously used. Among them, polyamide resins, polyester resins, polyurethane resins, polyolefin resins and fluorine resins are suitably used. In addition, the material that constitutes each tubular member may be the same as or different from the material that constitutes another tubular member. If the material that constitutes the second tubular member 20 and the material that constitutes the other tubular members 10, 30, 40 are the same, the joining property of the second tubular member 20 and the other tubular members in the process to be described later is satisfactory, and hence the materials configuring the first tubular member 10 and the second tubular member 20 can be made the same. Furthermore, for example, the strength on the proximal side of the in-vivo indwelling tube 1 can be increased and high flexibility can be obtained on the distal side by making the material that constitutes the third tubular member 30 and the fourth tubular member 40 as a material having higher strength than the material that constitutes the first tubular member 10.

As shown in FIG. 2, the first process is a process of placing the distal end 22 of the second tubular member 20 in the lumen of the first tubular member 10. In the first process, the placement of the distal end 22 of the second tubular member 20 in the lumen of the first tubular member 10 may insert the distal end 22 of the second tubular member 20 to the lumen from the proximal end 11 side of the first tubular member 10. In order to facilitate the placement of the distal end 22 of the second tubular member 20 in the lumen of the first tubular member 10, the inner diameter of the proximal end 11 of the first tubular member 10 is preferably larger than the inner diameter of the distal end 22 of the second tubular member 20.

The thickness of the first tubular member 10 is preferably 0.2 mm or more and 0.6 mm or less. The inner diameter of the first tubular member 10 may be constant over the entire axial direction or may differ depending on the axial positions. The inner diameter of the proximal end 11 of the first tubular member 10 is preferably greater than the outer diameter of the distal end of the second tubular member 20. With the inner diameter of the proximal end 11 of the first tubular member 10 being configured in this manner, the distal end 22 of the second tubular member 20 can be easily placed in the lumen of the first tubular member 10. Moreover, the second process to be described later can be easily performed. The diameter of the proximal end 11 of the first tubular member 10 can be expanded to be greater than the outer diameter of the distal end of the second tubular member 20. Furthermore, the diameter of the distal end 22 of the second tubular member 20 can be reduced to be smaller than the inner diameter of the proximal end of the first tubular member 10. The diameter-expanded portion and the diameter-reduced portion may have a stepped shape or a tapered shape.

Furthermore, in the first process, the placement of the distal end 22 of the second tubular member 20 into the lumen of the first tubular member 10 may be such that a slit (not shown in the figure) is formed at the proximal end portion of the first tubular member 10 and the first tubular member 10 is placed over the distal end 22 of the second tubular member 20. Since the portion of the first tubular member 10 provided with the slit expands radially outward, the distal side of the second tubular member 20 can be easily inserted to the portion of the first tubular member 10 provided with the slit.

Here, the length of the slit is preferably a length within 10 mm from the proximal end 11 of the first tubular member 10. In order to allow the first tubular member 10 to be easily expanded radially outward, the slit preferably penetrates the inside and the outside of the first tubular member 10. The slit is preferably provided along the axial direction of the first tubular member 10. As the slit is provided in the first tubular member 10 in this manner, the portion of the first tubular member 10 where the slit is provided can be easily expanded radially outward.

The method of providing the slit in the first tubular member 10 is not particularly limited, and for example, cutting by a rotary blade, a tube cutter, and a laser can be used.

One or more slits may be provided in the first tubular member 10, and for example, two or more, three or more, or five or less are acceptable. When a plurality of slits are provided in the first tubular member 10, each of the slits are preferably arranged at equal intervals in the circumferential direction of the first tubular member 10. The portion of the first tubular member 10 where the slit is provided can be easily expanded radially outward by providing the plurality of slits in the first tubular member 10 in this manner.

The inner diameter of the second tubular member 20 may be constant over the entire axial direction or may differ depending on the axial position. The inner diameter of the second tubular member 20 may be the same size as or may be different from the inner diameter of the distal end 22 of the second tubular member 20. If the inner diameter of the second tubular member 20 and the inner diameter of the proximal end of the first tubular member 10 have the same size, the discharging function of the body fluid of the in-vivo indwelling tube 1 is unlikely to be impeded. Therefore, the inner diameter of the second tubular member 20 is preferably equal to or close to the inner diameter of the proximal end of the first tubular member 10.

The thickness of the second tubular member 20 is preferably smaller than the thicknesses of the first tubular member 10, the third tubular member 30 described later, and the fourth tubular member 40. The thickness of the second tubular member 20 is preferably 0.2 times or more of the thickness of the first tubular member 10. Since the thickness of the second tubular member 20 is configured in this manner, the work of placing the second tubular member 20 on another member such as the first tubular member 10 can be easily performed. In addition, if the thickness of the second tubular member 20 is thinner, the step difference at the connecting portion between the second tubular member and the other members is reduced, and the delivery performance of the in-vivo indwelling tube 1 is improved.

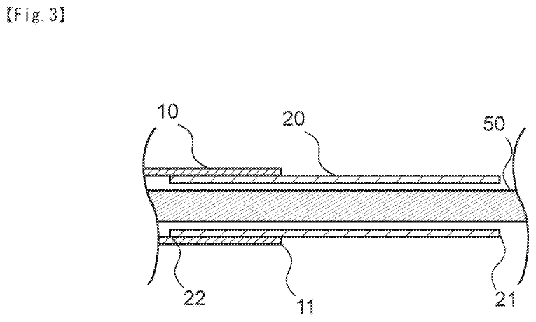

As shown in FIG. 3, the second process is a process of joining the first tubular member 10 and the second tubular member 20. The portion including the proximal end 11 of the first tubular member 10 and the portion including the distal end 22 of the second tubular member 20 are joined together. Specifically, the inner surface of the proximal end portion of the first tubular member 10 and the outer surface of the distal end portion of the second tubular member 20 placed in the lumen of the first tubular member 10 are joined and fixed. As a method of joining the first tubular member 10 and the second tubular member 20, for example, welding by heat or high frequency, adhesion by adhesives, and the like are mentioned. Among them, the first tubular member 10 and the second tubular member 20 are preferably welded. The first tubular member 1 and the second tubular member 20 can be firmly joined by welding the first tubular member 10 and the second tubular member 20.

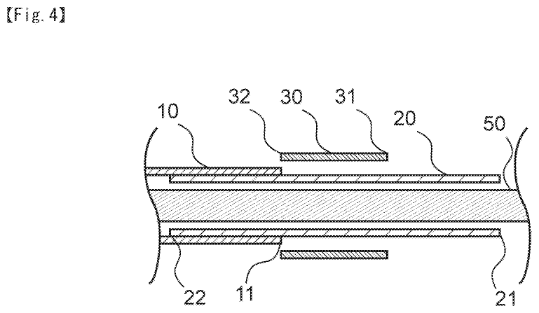

As shown in FIG. 4, the third process is a process of arranging the second tubular member 20 in the lumen of the third tubular member 30.

Here, the second tubular member 20 is a tubular member extending in the perspective direction, and is used to connect the first tubular member 10, the third tubular member 30, and the fourth tubular member 40 described later. The third tubular member 30 is a tubular member extending in the perspective direction, and can be used to connect the first tubular member 10, the second tubular member 20, and the fourth tubular member 40 described later. The proximal end 31 of the third tubular member 30 is preferably in contact with the distal end 42 of the fourth tubular member.

The length in an axial direction from the distal end 32 to the proximal end 31 of the third tubular member 30 is shorter than the length in an axial direction from the distal end 22 to the proximal end 21 of the second tubular member 20. The placement of the second tubular member 20 in the lumen of the third tubular member 30 can be performed through a method similar to the first process of placing the distal end 22 of the second tubular member 20 in the lumen of the first tubular member 10, and among them, it is preferable to insert the proximal end 21 of the second tubular member 20 from the distal end 32 side of the third tubular member 30. The second tubular member 20 can be easily and reliably placed in the lumen of the third tubular member 30 by placing the second tubular member 20 in the lumen of the third tubular member 30 in such a manner.

The inner diameter of the third tubular member 30 is preferably larger than the outer diameter of the second tubular member 20. As the inner diameter of the third tubular member 30 is configured in such a manner, the second tubular member 20 can be easily placed in the lumen of the third tubular member 30. In addition, the fourth process described later can be easily performed.

The outer diameter of the third tubular member 30 is preferably 0.9 times or more of the outer diameter of the first tubular member 10, and preferably 1.3 times or less of the outer diameter of the first tubular member 10. Since the outer diameter of the third tubular member 30 is configured in this way, the step difference between the first tubular member 10 and the third tubular member 30 is small when performing the fourth process to be described later.

The thickness of the third tubular member 30 may be the same as or different from the thickness of the first tubular member 10. The thickness of the third tubular member 30 is preferably 0.6 times or more of the thickness of the first tubular member 10, and preferably 1.3 times or less of the thickness of the first tubular member 10. Since the thickness of the third tubular member 30 is configured in this manner, the difference between the joining strength between the first tubular member 10 and the second tubular member 20 and the joining strength between the second tubular member 20 and the third tubular member 30 becomes small when performing the fourth process to be described later. The total thickness of the thickness of the third tubular member 30 and the thickness of the second tubular member 20 is most preferably equal to the thickness of the first tubular member 10.

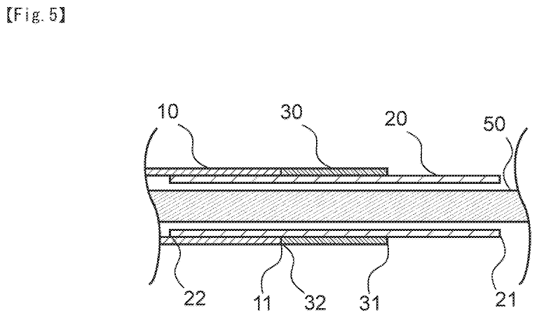

As shown in FIG. 5, the fourth process is a process of joining the second tubular member 20 and the third tubular member 30. The outer surface of the second tubular member 20 and the inner surface of the third tubular member 30 are joined and fixed to each other. As a method of joining the second tubular member 20 and the third tubular member 30, although it can be carried out by a method similar to the second process of joining the first tubular member 10 and the second tubular member 20, among them, the members are preferably joined by welding. The second tubular member 20 and the third tubular member 30 can be strongly joined by joining the second tubular member 20 and the third tubular member 30 by welding. Moreover, the second process and the fourth process are preferably carried out with the same heating process. The variation in the joining strength between the first tubular member 10, the third tubular member 30, and the second tubular member 20 can be suppressed by performing the second process and the fourth process with the same heating process.

In the fourth process, the first tubular member 10 and the third tubular member 30 may be joined. Specifically, portions including the proximal end 11 of the first tubular member 10 and the distal end 32 of the third tubular member 30 are joined together. The end face of the proximal end 11 of the first tubular member 10 and the end face of the distal end 32 of the third tubular member 30 may be joined, and the outer surface of the proximal end portion of the first tubular member 10 and the inner surface of the distal end portion of the third tubular member 30 may be joined. Since the first tubular member 10 and the third tubular member 30 are arranged adjacent to each other, the second tubular member 20 and the third tubular member 30 can be efficiently joined in the process of joining them.

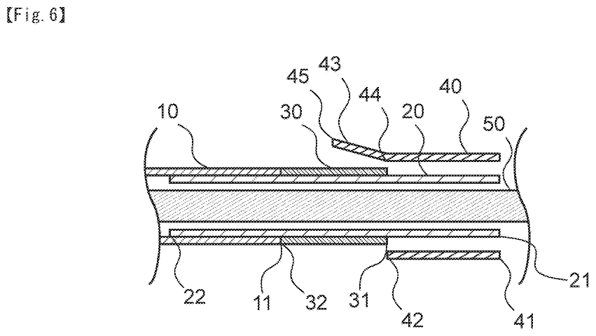

As shown in FIG. 6, the fifth process is a process of placing the proximal end 21 of the second tubular member 20 in the lumen on the distal side of the fourth tubular member 40. The fourth tubular member 40 is an axially extending tubular member, and includes a flap 43 having a base end 44 on the proximal side and a free end 45 on the distal side. The base end 44 is a base point at which the flap 43 rises from the fourth tubular member 40, and the free end 45 is a tip of the flap 43 raised from the fourth tubular member 40. The placement of the proximal end 21 of the second tubular member 20 in the lumen on the distal side of the fourth tubular member 40 can be performed through a method similar to the first process and the third process, and among them, the proximal end 21 of the second tubular member 20 is preferably inserted and arranged in the lumen on the proximal side of the base end 44 of the flap 43 of the fourth tubular member 40. By arranging the proximal end 21 of the second tubular member 20 in the lumen on the distal side of the fourth tubular member 40 in this manner, the placement can be performed easily and reliably.

The fourth tubular member 40 uses, similar to the first tubular member 10, the second tubular member 20, and the third tubular member 30, for example, one in which the flap 43 is formed on the resin tube molded by extrusion molding. The flap 43 is formed by, for example, joining a separate member to be the flap 43 to the resin tube, separating the portion other than the portion to be the flap 43 to form the flap 43 at the distal end of the resin tube, and the like.

The material that constitutes the flap 43 of the fourth tubular member 40 and the material that constitutes the portion other than the flap 43 may be the same or different. If the material that constitutes the portion other than the flap 43 and the material that constitutes the flap 43 are the same, the portion other than the flap 43 and the flap 43 can be strongly joined. Furthermore, if the material that constitutes the portion other than the flap 43 and the material that constitutes the flap 43 are different, for example, the portion other than the flap 43 is made flexible using a soft material and the strength of the flap 43 is increased using a hard material for the flap 43.

The type A durometer hardness of the material that constitutes the fourth tubular member 40 is preferably higher than the type A durometer hardness of the material that constitutes the first tubular member 10, the second tubular member 20, and the third tubular member 30. Type A durometer hardness can be measured by a method conforming to JIS K7215. The rigidity of the flap 43 can be increased and the function of fixing the in-vivo indwelling tube 1 in the lumen in the living body can be enhanced by making the hardness of the material that constitutes the fourth tubular member 40 higher than the hardness of the materials that constitute the first tubular member 10, the second tubular member 20, and the third tubular member 30.

The inner diameter of the fourth tubular member 40 is preferably larger than the outer diameter of the second tubular member 20. This facilitates the placement of the second tubular member 20 in the lumen of the fourth tubular member 40. Furthermore, the sixth process described later can be easily performed.

The outer diameter of the fourth tubular member 40 is preferably 0.7 or more times the outer diameter of the third tubular member 30, and preferably 1.3 or less times the outer diameter of the third tubular member 30. Since the outer diameter of the fourth tubular member 40 is configured in this way, the step difference between the third tubular member 30 and the fourth tubular member 40 is small in the finish and the low invasive in-vivo indwelling tube 1 can be obtained.

The thickness of the fourth tubular member 40 may be the same as or different from the thickness of the third tubular member 30. The thickness of the fourth tubular member 40 is preferably 0.7 times or more of the thickness of the third tubular member 30, and preferably 2.0 times or less of the thickness of the third tubular member 30. Since the thickness of the fourth tubular member 40 is configured in this manner, the difference between the joining strength between the first tubular member 10 and the second tubular member 20 and the joining strength between the third tubular member 30 and the fourth tubular member 40 becomes small when performing the sixth process to be described later.

The thickness of the flap 43 of the fourth tubular member 40 may be the same as or different from the thickness of at least either the first tubular member 10 or the third tubular member 30. Here, the thickness of the flap 43 refers to the thickness of the base end 44 of the flap 43. The thickness of the flap 43 does not include the thickness of the portion forming the lumen of the fourth tubular member 40. If the thickness of the flap 43 of the fourth tubular member 40 is thick, the strength of the flap 43 is increased, and the proximal end 102 of the in-vivo indwelling tube 1 is prevented from entering into the lumen of the living body such as the bile duct where constriction has occurred. If the thickness of the flap 43 of the fourth tubular member 40 is thin, the delivery performance of the in-vivo indwelling tube 1 is enhanced. It is preferable to adjust the thickness of one part or all of each of the tubular members 10, 20, 30, 40 in accordance with the size, required strength, and the like of the in-vivo indwelling tube 1.

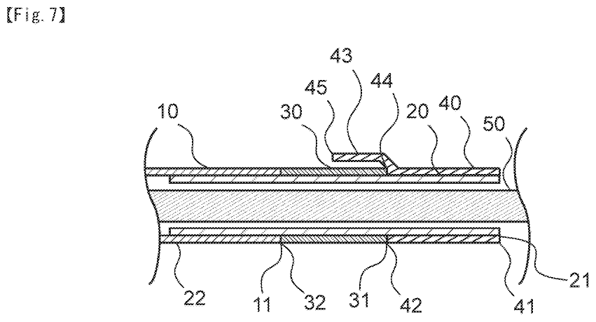

As shown in FIG. 7, the sixth process is a process of joining the second tubular member 20 and the fourth tubular member 40, and joins the outer surface of the second tubular member 20 and the inner surface on the proximal side of the base end 44 of the flap 43 of the fourth tubular member 40. The joining may be, for example, welding by heat or high frequency, adhesion by an adhesive, and the like. Among them, joining by welding is preferable. The second tubular member 20 and the fourth tubular member 40 can be strongly joined by joining the second tubular member 20 and the fourth tubular member 40 by welding. Furthermore, the second process and the fourth process are preferably carried out by the same heating process, and the second process, the fourth process, and the sixth process are more preferably carried out by the same heating process. The variation in the joining strength of the first tubular member 10 and the second tubular member 20, and the third tubular member 30 and the second tubular member 20 can be suppressed by performing the second process and the fourth process by the same heating process. Moreover, the variation in the joining strength of the first tubular member 10 and the second tubular member 20, the third tubular member 30 and the second tubular member 20, and the fourth tubular member 40 and the second tubular member 20 by performing the second process, the fourth process, and the sixth process by the same heating process.

In the sixth process, the third tubular member 30 and the fourth tubular member 40 may be joined. Specifically, portions including the proximal end 31 of the third tubular member 30 and the distal end 42 of the fourth tubular member 40 are joined together. The end face of the proximal end 31 of the third tubular member 30 and the end face of the distal end 42 of the fourth tubular member 40 may be joined, and the outer surface of the proximal end portion of the third tubular member 30 and the inner surface of the distal end portion of the fourth tubular member 40 may be joined. Since the third tubular member 30 and the fourth tubular member 40 are arranged adjacent to each other, the second tubular member 20 and the fourth tubular member 30 can be efficiently joined in the process of joining them.

A process of arranging a core material 50 in the lumen of the second tubular member 20 is preferably included before the first process. The core material 50 is a circular column shaped member extending in the axial direction, and is preferably longer than the length in an axial direction of the second tubular member 20. In order to arrange the core material 50 in the lumen of the second tubular member 20, for example, the core material 50 may be inserted into the distal end 22 or the proximal end 21 of the second tubular member 20, or the second tubular member 20 in which is a cut is formed radially outward of the core material 50 may be placed over. The axial position of each member can be easily aligned accurately in the first process, the third process, and the fifth process by arranging the core material 50 in the lumen of the second tubular member 20, so that the arrangement can be easily performed, the joining process is facilitated in the second process, the fourth process, and the sixth process, and joining can be reliably performed.

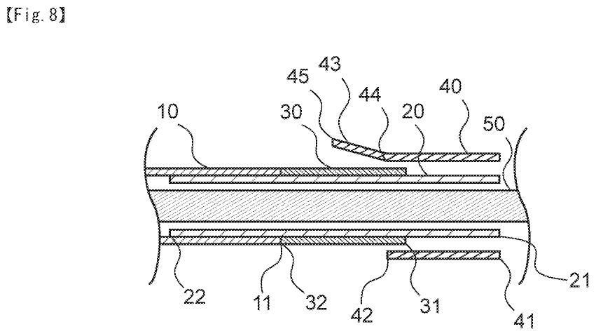

As shown in FIG. 8, the proximal end 31 of the third tubular member 30 is preferably inserted and arranged to the proximal side of the base end 44 of the flap 43 of the fourth tubular member 40 before the sixth process. The outer surface of the proximal end portion of the third tubular member 30 and the inner surface of a portion on the proximal side of the base end 44 of the flap 43 of the fourth tubular member 40 can be abutted by arranging the third tubular member 30 and the fourth tubular member 40 in this manner. A larger diameter portion (described later) in which the third tubular member 30 and the fourth tubular member 40 overlap with each other on the second tubular member 20 is formed by inserting the proximal end 31 of the third tubular member 30 on the proximal side of the base end 44 of the flap 43 of the fourth tubular member 40 before the sixth process. Therefore, a hole does not form in the vicinity of the base end 44 of the flap 43, and a lesion such as a cancer cell can be prevented from entering the lumen of the in-vivo indwelling tube 1 through the hole.

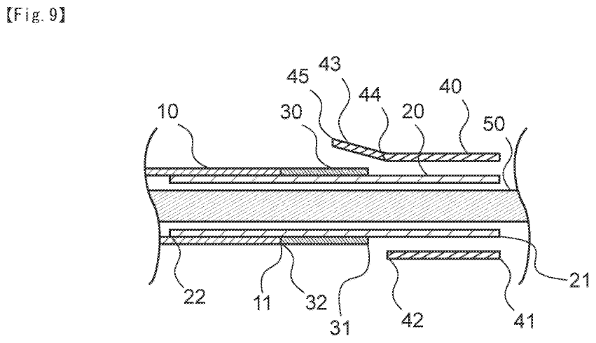

Furthermore, as shown in FIG. 9, the proximal end 31 of the third tubular member 30 is preferably arranged on the distal side of the base end 44 of the flap 43 of the fourth tubular member 40 before the sixth process. A state in which a gap is formed between the proximal end 31 of the third tubular member 30 and the base end 44 of the flap 43 of the fourth tubular member 40 is obtained by arranging the third tubular member 30 and the fourth tubular member 40 in this manner. By arranging the proximal end 31 of the third tubular member 30 on the distal side of the base end 44 of the flap 43 of the fourth tubular member 40 before the sixth process, a smaller diameter portion (to be described later) in which the second tubular member 20 is not covered by either the third tubular member 30 or the fourth tubular member 40 is formed, the inner wall of the conduit and the flap 43 are less likely to make contact when inserting the in-vivo indwelling tube 1 into the conduit of the endoscope, and the delivery performance of the in-vivo indwelling tube 1 is enhanced.

A process of cutting the distal side or the proximal side of the in-vivo indwelling tube may be added in a process after the above-mentioned joining process. The overall length of the in-vivo indwelling tube and the position from the end portion of the flap 43 can be controlled by the cutting process. In addition, the end face can be flattened by the cutting process. The length of each tubular member of the in-vivo indwelling tube is preferably as follows. First tubular member: 300 mm or more and 1800 mm or less, second tubular member: 10 mm or more and 500 mm or less, third tubular member: 10 mm or more and 30 mm or less, fourth tubular member: 4 mm or more and 20 mm or less. The base end 44 of the flap 43 is preferably provided in a region within 10 mm from the distal end 42 of the fourth tubular member 40.

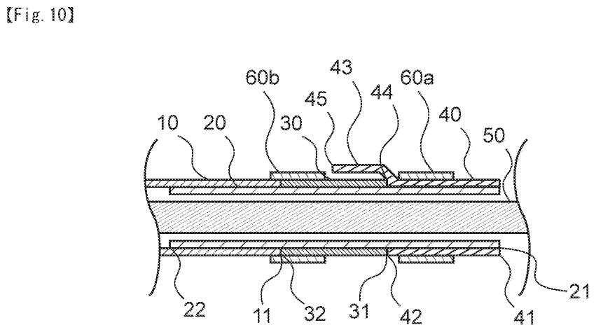

As shown in FIG. 10, after the sixth process, a supporting member 60 may be arranged on the proximal side of the base end 44 of the flap 43 of the fourth tubular member 40 and on the radially outward of at least either the distal side of the base end 44 of the flap 43 of the fourth tubular member 40 or the proximal side of the midpoint of the first tubular member 10. Here, the supporting member 60 provided on the proximal side of the base end 44 of the flap 43 of the fourth tubular member 40 is referred to as the proximal side first supporting member 60a, and the supporting member 60 provided on the distal side of the base end 44 of the flap 43 of the fourth tubular member 40 and on the proximal side of the midpoint of the first tubular member 10 is referred to as a proximal side second supporting member 60b. When stress is applied to the base end 44 of the flap 43 of the fourth tubular member 40 by arranging the proximal side first supporting member 60a on the proximal side of the base end 44 of the flap 43 of the fourth tubular member 40, the base end 44 can be prevented from tearing and the flap 43 from breaking. Furthermore, by arranging the proximal side second supporting member 60b on the distal side of the base end 44 of the flap 43 of the fourth tubular member 40 and on the proximal side of the midpoint of the first tubular member 10, the strength of the portion, where the proximal side second supporting member 60b is arranged, of the in-vivo indwelling tube 1 can be increased, and the pushability of the in-vivo indwelling tube 1 can be enhanced.

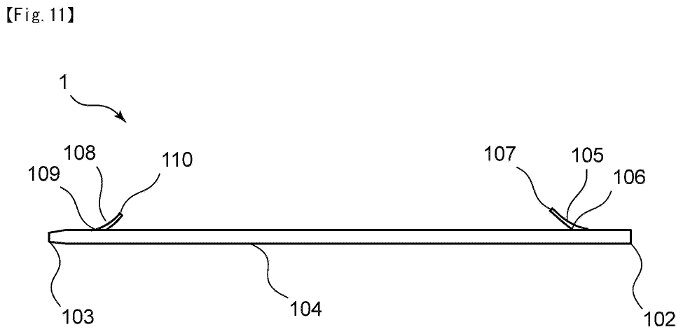

Embodiments of the in-vivo indwelling tube of the present invention will be described with reference to the drawings. As shown in FIG. 11, the in-vivo indwelling tube 1 includes a tubular member 104 having a proximal side and a distal side, a proximal flap 105 having a base end 106 on the proximal side and a free end 107 on the distal side, on the proximal side of the tubular member 104, and a distal flap 108 having a base end 109 on the distal side and a free end 110 on the proximal side, on the distal side of the tubular member 104. The in-vivo indwelling tube 1 has a proximal end 102 and a distal end 103 and extends axially. The base end 106 is a base point at which the proximal flap 105 rises from the tubular member 104, and the free end 107 is a tip of the proximal flap 105 raised from the tubular member 104. The base end 109 is a base point at which the distal flap 108 rises from the tubular member 104, and the free end 110 is a tip of the distal flap 108 raised from the tubular member 104.

The inner diameter of the tubular member 104 may be constant over the entire axial direction or may differ depending on the axial position.

The outer diameter of the distal end 103 of the tubular member 104 is preferably smaller than the average outer diameter of the tubular member 104 between the position of the tubular member 104 corresponding to the free end 110 of the distal flap 108 and the position of the tubular member 104 corresponding to the free end 107 of the proximal flap 105. The outer diameter of the tubular member 104 may be reduced in a tapered manner towards the distal end 103 at the distal end portion. Since the outer diameter of the distal end 103 of the tubular member 104 is reduced, the in-vivo indwelling tube 1 can easily pass through the constricted portion or the occluded portion of the lumen in the living body.

The thickness of the tubular member 104 can be appropriately set according to the necessary strength and flexibility, but is preferably 0.2 mm or more and 0.6 mm or less. The thicknesses of the proximal flap 105 and the distal flap 108 are each preferably 0.2 mm or more and 0.6 mm or less. The wall thicknesses of the proximal flap 105 and the distal flap 108 each may be the same or may be different. If different, and if the thickness of the proximal flap 105 is thinner than the thickness of the distal flap 108, the proximal flap 105 is less likely to interfere with the inner wall of the conduit of the endoscope, thus enhancing the delivery performance. If the thickness of the proximal flap 105 is thicker than the thickness of the distal flap 108, the strength of the proximal flap 105 is increased and the effect of preventing the proximal end 102 of the in-vivo indwelling tube 1 from entering the lumen in the living body can be enhanced.

The thickness of the proximal end 102 of the tubular member 104 is preferably thicker than the average thickness of the tubular member 104 between the position of the tubular member 104 corresponding to the free end 110 of the distal flap 108 and the position of the tubular member 104 corresponding to the free end 107 of the proximal flap 105. With this thickness of the proximal end 102 of the tubular member 104, the pushability of the in-vivo indwelling tube 1 can be improved. The end face of the proximal end 102 is preferably flat. Thus, the strength of the end face of the proximal end 102 of the in-vivo indwelling tube 1 becomes constant, and the pushability of the in-vivo indwelling tube 1 can be improved. Furthermore, the end face of the proximal end 102 may be chamfered at its outer periphery so as not to damage the body cavity.

Examples of a material that constitutes the tubular member 104, the proximal flap 105, and the distal flap 108 include polyamide resins, polyester resins, polyurethane resins, polyolefin resins, fluorine resins, vinyl chloride resins, silicone resins, natural rubbers, and the like. Only one kind of resin may be used or two or more kinds of resins may be simultaneously used. Among them, polyamide resins, polyester resins, polyurethane resins, polyolefin resins and fluorine resins are suitably used. The material that constitutes the tubular member 104, the proximal flap 105, and the distal flap 108 may be the same or may be different. If the material that constitutes the tubular member 104, the proximal flap 105, and the distal flap 108 is the same, the overall strength and flexibility of the in-vivo indwelling tube 1 become uniform. Furthermore, the in-vivo indwelling tube 1 in which the retention of the proximal flap 105 and the distal flap 108 is high but the flexibility of the tubular member 104 is maintained is obtained by having the material that constitutes the proximal flap 105 and the distal flap 108 as a material having a higher hardness than the material that constitutes the tubular member 104.

The proximal flap 105 extends axially and radially outward from the proximal side to the distal side. The distal flap 108 extends axially and radially outward from the distal side to the proximal side. One or more of the proximal flap 105 and the distal flap 108 may be provided, for example, two or more, three or more, or five or less. When a plurality of proximal flaps 105 or a plurality of distal flaps 108 are provided, the flaps 105, 108 are preferably arranged at equal intervals in the circumferential direction of the tubular member 104. With the plurality of proximal flaps 105 arranged in this manner, the effect of preventing the proximal end 102 of the in-vivo indwelling tube 1 from entering the lumen in the living body can be enhanced. With the plurality of distal flaps 108 arranged in this manner, the effect of preventing the in-vivo indwelling tube 1 from falling out of the lumen in the living body can be enhanced. When a plurality of flaps are provided, the length from the base end to the free end of the flap to be described later and the width and thickness of the flap may all be the same or different. For example, if the length, width, and thickness of each flap are the same, production becomes easy. Furthermore, the strength of each flap can be changed by changing the length, width, and thickness of each flap. Specific examples include raising the strength of the flap at a portion where load is likely to be applied and breakage may occur, and decreasing the strength of the flap at a portion where flexibility is required.

The length from the base end 106 to the free end 107 of the proximal flap 105 and the length from the base end 109 to the free end 110 of the distal flap 108 are not particularly limited, but are preferably 4 mm or more and 15 mm or less.

The thickness of the proximal flap 105 is not particularly limited, but may be thinner or thicker than the thickness of the proximal end of the tubular member 104. The lumen in the living body that comes into contact with the proximal flap 105 can be made to be less likely to be damaged by making the thickness of the proximal flap 105 thinner than the thickness of the proximal end of the tubular member 104. The strength of the proximal flap 105 can be increased by making the thickness of the proximal flap 105 thicker than the thickness of the proximal end of the tubular member 104. Furthermore, the thickness of the proximal flap 105 may be constant or may be different from the base end 106 to the free end 107. For example, the proximal flap may include a portion where the thickness decreases from the base end 106 toward the free end 107 of the proximal flap 105.

The thickness of the distal flap 108 is not particularly limited, but is preferably equal to or thinner than the thickness of the proximal end 102 of the tubular member 104. The distal flap 108 can be prevented from penetrating or damaging the lumen in the living body by making the thickness of the distal flap 108 thinner than the thickness of the proximal end 102 of the tubular member 104. Furthermore, the thickness of the distal flap 108 is preferably thicker than the average thickness of the tubular member 104 between the position of the tubular member 104 corresponding to the free end 110 of the distal flap 108 and the position of the tubular member 104 corresponding to the free end 107 of the proximal flap 105. This thickness of the distal flap 108 can enhance the retention of the distal flap 108 in the lumen in the living body. The thickness of the distal flap 108 may be constant or may be different from the base end 109 to the free end 110 of the distal flap 108.

The thickness and length of the proximal flap 105 may be the same as or different from the thickness and length of the distal flap 108. For example, by making the length of the proximal flap 105 longer than the length of the distal flap 108, the proximal flap 105 can be greatly opened radially outward, and the effect of preventing the proximal end 102 of the in-vivo indwelling tube 1 from entering the lumen in the living body where constriction has occurred can be enhanced. Moreover, for example, by making the length of the distal flap 108 longer than the length of the proximal flap 105, the opening size of the distal flap 108 can be increased, and the retention of the in-vivo indwelling tube 1 to the lumen in the living body can be increased.

The material that constitutes the proximal flap 105 and the distal flap 108 is not particularly limited, and those listed as the resin that constitutes the tubular member 104 can be used. The material that constitutes the proximal flap 105 may be the same as or different from the material that constitutes the tubular member 104. If the material that constitutes the flaps 105, 108 and the material that constitutes the tubular member 104 are the same, the joinability between the tubular member 104 and the flaps 105, 108 is improved, and the flaps 105, 108 are easily provided on the tubular member 104. If the material that constitutes the flaps 105, 108 and the material that constitutes the tubular member 104 are different, for example, a soft material is used for the material that constitutes the tubular member 104 so that the in-vivo indwelling tube 1 in which the flexibility is high and the strength of the flaps 105, 108 is high is obtained.

The type A durometer hardness of the material that constitutes the proximal flap 105 or the distal flap 108 is preferably higher than the average type A durometer hardness of the material that constitutes the tubular member 104 between the position of the tubular member 104 corresponding to the free end 110 of the distal flap 108 and the position of the tubular member 104 corresponding to the free end 107 of the proximal flap 105. Type A durometer hardness can be measured by a method conforming to JIS K7215. The rigidity of the proximal flap 105 can be increased and the function of preventing the proximal end 102 of the in-vivo indwelling tube 1 from entering the lumen in the living body can be enhanced by having the hardness of the material that constitutes the proximal flap 105 higher than the hardness of the tubular member 104. Furthermore, the rigidity of the distal flap 108 can be increased and the function of fixing the in-vivo indwelling tube 1 in the lumen in the living body can be enhanced by having the hardness of the material that constitutes the distal flap 108 higher than the hardness of the tubular member 104.

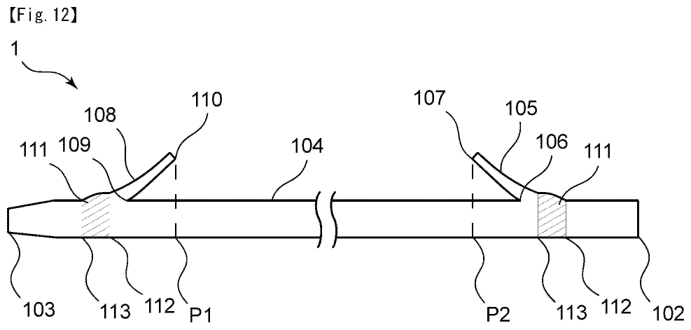

As shown in FIG. 12, the in-vivo indwelling tube 1 preferably includes a larger diameter portion 111 in which the maximum outer diameter is larger than the average outer diameter of the tubular member 104 between the position P1 of the tubular member 104 corresponding to the free end 110 of the distal flap 108 and the position P2 of the tubular member 104 corresponding to the free end 107 of the proximal flap 105 on at least either the proximal side of the base end 106 of the proximal flap 105 or the distal side of the base end 109 of the distal flap 108. The position P1 of the tubular member 104 corresponding to the free end 110 of the distal flap 108 is the position where the free end 110 of the distal flap 108 comes into contact with the tubular member 104 when the distal flap 108 is arranged along the tubular member 104 and the distal flap 108 is closed. The position P2 of the tubular member 104 corresponding to the free end 107 of the proximal flap 105 is the position where the free end 107 of the proximal flap 105 comes into contact with the tubular member 104 when the proximal flap 105 is arranged along the tubular member 104 and the proximal flap 105 is closed. The strength of the proximal flap 105 can be enhanced and the breakage of the proximal flap 105 can be prevented by including the larger diameter portion 111 on the proximal side of the base end 106 of the proximal flap 105. Similarly, the strength of the distal flap 108 can be enhanced by including the larger diameter portion 111 on the distal side of the base end 109 of the distal flap 108.

In order to form the larger diameter portion 111 on the proximal side of the base end 106 of the proximal flap 105, for example, the proximal end 31 of the third tubular member 30 is to be arranged on the proximal side of the base end 44 of the flap 43 of the fourth tubular member 40 before the sixth process. The larger diameter portion 111 is formed on the proximal side of the base end 106 of the proximal flap 105 of the in-vivo indwelling tube 1 by producing the in-vivo indwelling tube 1 in such a manner. In addition, a hole does not form in the vicinity of the base end 106 of the proximal flap 105, and a lesion such as a cancer cell can be prevented from entering the lumen of the in-vivo indwelling tube 1 through the hole.

In order to form the larger diameter portion 111 on the distal side of the base end 109 of the distal flap 108, for example, the distal end 32 of the third tubular member 30 is to be arranged on the distal side of the base end 44 of the flap 43 of the fourth tubular member 40 before the sixth process. The larger diameter portion 111 is formed on the distal side of the base end 109 of the distal flap 108 of the in-vivo indwelling tube 1 by producing the in-vivo indwelling tube 1 in such a manner. In addition, a hole does not form in the vicinity of the base end 109 of the distal flap 108, and a lesion such as a cancer cell can be prevented from entering the lumen of the in-vivo indwelling tube 1 through the hole.

The maximum outer diameter of the larger diameter portion 111 is preferably 1.05 times or more than the average outer diameter of the tubular member 104 between the position P1 and the position P2 of the tubular member 104, and more preferably 1.07 times or more preferably, 1.1 times or more. The strength of the proximal flap 105 can be increased by setting the lower limit value of the maximum outer diameter of the larger diameter portion 111 in this manner. Furthermore, the maximum outer diameter of the larger diameter portion 111 is preferably 1.3 times or less than the average outer diameter of the tubular member 104 between the position P1 and the position P2 of the tubular member 104, and is more preferably 1.25 times or less and more preferably 1.2 times or less. Flexibility can be provided to the vicinity of the proximal flap 105 of the in-vivo indwelling tube 1 by setting the upper limit value of the maximum outer diameter of the larger diameter portion 111 in this manner.

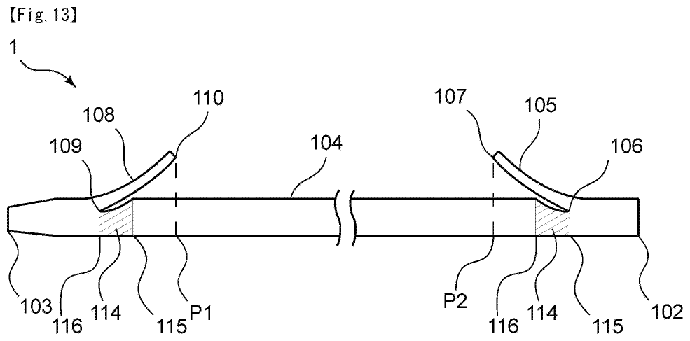

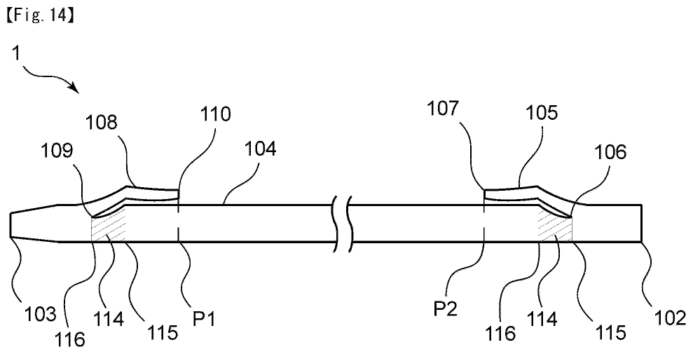

As shown in FIG. 13, the in-vivo indwelling tube 1 preferably includes a smaller diameter portion 114 in which the minimum outer diameter is smaller than the average outer diameter of the tubular member 104 between the position P1 of the tubular member 104 corresponding to the free end 110 of the distal flap 108 and the position P2 of the tubular member 104 corresponding to the free end 107 of the proximal flap 105. As shown in FIG. 14, the smaller diameter portion 114 is preferably provided on at least either the position on the distal side of the base end 106 of the proximal flap 105 and where the free end 107 of the proximal flap 105 when the proximal flap 105 is in the closed state is on the distal side of the distal end 116 of the smaller diameter portion 114 or the position on the proximal side of the base end 109 of the distal flap 108 and where the free end 110 of the distal flap 108 when the distal flap 108 is in the closed state is on the proximal side of the proximal end 115 of the smaller diameter portion 114. The closed state of the proximal flap 105 refers to a state in which the proximal flap 105 is arranged along the tubular member 104 and the proximal flap 105 is closed. The closed state of the distal flap 108 refers to a state in which the distal flap 108 is arranged along the tubular member 104 and the distal flap 108 is closed. The smaller diameter portion 114 may have a hole to be described later so that the minimum outer diameter may be smaller than the average outer diameter of the tubular member 104 between the position P1 of the tubular member 104 corresponding to the free end 110 of the distal flap 108 and the position P2 of the tubular member 104 corresponding to the free end 107 of the proximal flap 105, or may not have a hole and may be reduced entirely in the radial direction so that the minimum outer diameter is smaller.

The outer diameter of the tubular member 104 in an area where the tubular member 104 and the proximal flap 105 overlap when the proximal flap 105 is closed can be reduced by including the smaller diameter portion 114 on the proximal side of the free end 107 of the proximal flap 105. The inner wall of the conduit is less likely to interference with the in-vivo indwelling tube 1 and smooth passing of the conduit is realized when passing the in-vivo indwelling tube 1 through the conduit of the endoscope and the like by reducing the outer diameter of the tubular member 104 in an area where the tubular member 104 and the proximal flap 105 overlap. Furthermore, the outer diameter of the tubular member 104 in an area where the tubular member 104 and the distal flap 108 overlap when the distal flap 108 is closed can be reduced by including the smaller diameter portion 114 on the distal side of the free end 110 of the distal flap 108.

In order to form the smaller diameter portion 114 on the distal side of the base end 106 of the proximal flap 105, for example, the proximal end 31 of the third tubular member 30 is to be arranged on the distal side of the base end 44 of the flap 43 of the fourth tubular member 40 before the sixth process. Thus, the smaller diameter portion 114 is formed on the distal side of the base end 106 of the proximal flap 105 of the in-vivo indwelling tube 1, and the outer diameter of an area where the tubular member 104 and the proximal flap 105 overlap can be reduced in a state where the proximal flap 105 is closed along the tubular member 104 by producing the in-vivo indwelling tube 1 in such a manner.

In order to form the smaller diameter portion 114 on the proximal side of the base end 109 of the distal flap 108, for example, the distal end 32 of the third tubular member 30 is to be arranged on the proximal side of the base end 44 of the flap 43 of the fourth tubular member 40 before the sixth process. Thus, the smaller diameter portion 114 is formed on the proximal side of the base end 109 of the distal flap 108 of the in-vivo indwelling tube 1, and the outer diameter of an area where the tubular member 104 and the distal flap 108 overlap can be reduced in a state where the distal flap 108 is closed along the tubular member 104 by producing the in-vivo indwelling tube 1 in such a manner.

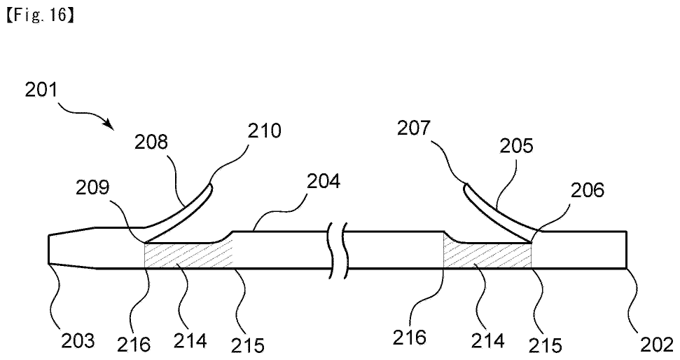

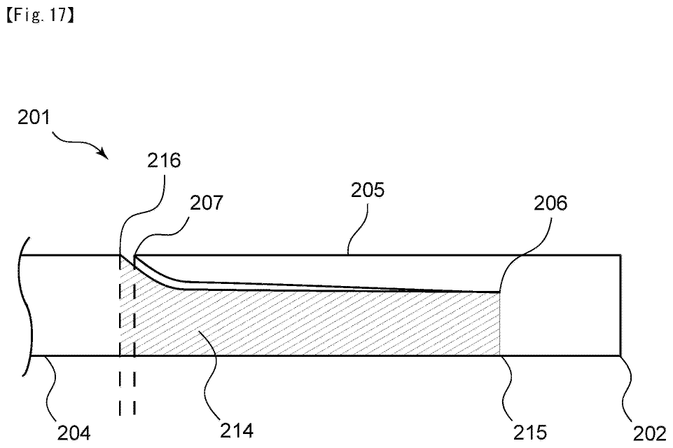

As shown in FIGS. 16 and 17, the conventional in-vivo indwelling tube 201 is formed with a cut on the proximal side of the tubular member 204 to form the proximal flap 205, and formed with a cut on the distal side to form the distal flap 208. Therefore, in a state where the proximal flap 205 is arranged along the tubular member 204 and the proximal flap 205 is closed, the free end 207 of the proximal flap 205 is located on the proximal side of the distal end 216 of the smaller diameter portion 214. Furthermore, in a state where the distal flap 208 is closed, the free end 210 of the distal flap 208 is located on the distal side of the proximal end 215 of the smaller diameter portion 214.

As shown in FIGS. 13 and 14, in a state where the proximal flap 105 is closed, the in-vivo indwelling tube 1 of the present invention has the free end 107 of the proximal flap 105 located on the distal side of the distal end 116 of the smaller diameter portion 114. Furthermore, in a state where the distal flap 108 is closed, the free end 110 of the distal flap 108 is located on the proximal side of the proximal end 115 of the smaller diameter portion 114. The length in an axial direction of the smaller diameter portion 114 is preferably shorter than at least either the length in an axial direction of the proximal flap 105 or the length in an axial direction of the distal flap 108.

When the smaller diameter portion 114 is on the distal side of the base end 106 of the proximal flap 105 and the free end 107 of the proximal flap 105 when the proximal flap 105 is closed is provided at a position on the distal side of the distal end 116 of the smaller diameter portion 114, the maximum outer diameter of the proximal end 115 of the smaller diameter portion 114 is preferably smaller than the maximum outer diameter of the distal end 116 of the smaller diameter portion 114. Such a maximum outer diameter of the smaller diameter portion 114 allows the outer diameter of the area where the tubular member 104 and the proximal flap 105 overlap to be further reduced when the proximal flap 105 is in the closed state.

When the smaller diameter portion 114 is on the proximal side of the base end 109 of the distal flap 108 and the free end 110 of the distal flap 108 when the distal flap 108 is closed is provided at a position on the proximal side of the proximal end 115 of the smaller diameter portion 114, the minimum outer diameter of the distal end 116 of the smaller diameter portion 114 is preferably smaller than the minimum outer diameter of the proximal end 115 of the smaller diameter portion 114. Such a maximum outer diameter of the smaller diameter portion 114 allows the outer diameter of the area where the tubular member 104 and the distal flap 108 overlap to be further reduced when the distal flap 108 is in the closed state.

The tubular member 104 may have a hole at the smaller diameter portion 114. The hole may be a through hole that communicates the lumen of the tubular member 104 and the exterior of the tubular member 104, or may be a recess on the tubular member 104 and a hole that does not communicate the lumen of the tubular member 104 and the exterior of the tubular member 104. Furthermore, a hole may be provided in the entire region of the smaller diameter portion 114. That is, the smaller diameter portion 114 may be a hole.

The cross-sectional area of the hole in the plane perpendicular to the depth direction of the hole is not particularly limited, but is preferably smaller than the maximum cross-sectional area of the lumen of the tubular member 104. If the hole has such a size, even if a lesion such as a cancer cell comes into contact with the hole, the lesion can be made less likely to enter the lumen of the in-vivo indwelling tube 1.

The shape of the hole is not particularly limited, and examples thereof include a circle, an ellipse, and a rectangle. Moreover, the length of the hole in the axial direction of the tubular member 104 may be shorter than the length from the distal end 116 to the proximal end 115 of the smaller diameter portion 114 when the smaller diameter portion 114 is on the distal side of the base end 106 of the proximal flap 105 and the free end 107 of the proximal flap 105 is provided at a position on the distal side of the distal end 116 of the smaller diameter portion 114 when the proximal flap 105 is in the closed state. The length of the hole in the direction orthogonal to the axial direction of the tubular member 104 may be shorter than the length in the direction orthogonal to the axial direction of the proximal flap 105. Such a shape of the hole allows the possibility of a lesion such as a cancer cell entering the lumen of the in-vivo indwelling tube 1 through the hole to be reduced. Furthermore, a hole may be provided in the entire region of the smaller diameter portion 114. That is, the smaller diameter portion 114 may be a hole.

Similarly, the length of the hole in the axial direction of the tubular member 104 may be shorter than the length from the proximal end 115 to the distal end 116 of the smaller diameter portion 114 when the smaller diameter portion 114 is on the proximal side of the base end 109 of the distal flap 108 and the free end 110 of the distal flap 108 is provided at a position on the proximal side of the proximal end 115 of the smaller diameter portion 114 when the distal flap 108 is in the closed state. The length of the hole in the direction orthogonal to the axial direction of the tubular member 104 is preferably shorter than the length in the direction orthogonal to the axial direction of the distal flap 108. Furthermore, a hole may be provided in the entire region of the smaller diameter portion 114. That is, the smaller diameter portion 114 may be a hole.

The position of the hole is not particularly limited, but when the smaller diameter portion 114 is on the distal side of the base end 106 of the proximal flap 105 and the free end 107 of the proximal flap 105 is provided at a position on the distal side of the distal end 116 of the smaller diameter portion 114 when the proximal flap 105 is in the closed state, the hole may be extended from the proximal end 115 to the distal end 116 of the smaller diameter portion 114 or a hole may be arranged on the proximal side of the midpoint between the distal end 116 and the proximal end 115 of the smaller diameter portion 114. If the position of the hole is in this manner, a lesion such as a cancer cell is less likely to enter the lumen of the in-vivo indwelling tube 1 through the hole.

Similarly, the hole may be extended from the proximal end 115 to the distal end 116 of the smaller diameter portion 114 or a hole may be arranged on the distal side of the midpoint between the proximal end 115 and the distal end 116 of the smaller diameter portion 114 when the smaller diameter portion 114 is on the proximal side of the base end 109 of the distal flap 108 and the free end 110 of the distal flap 108 is provided at a position on the proximal side of the proximal end 115 of the smaller diameter portion 114 when the distal flap 108 is in the closed state.

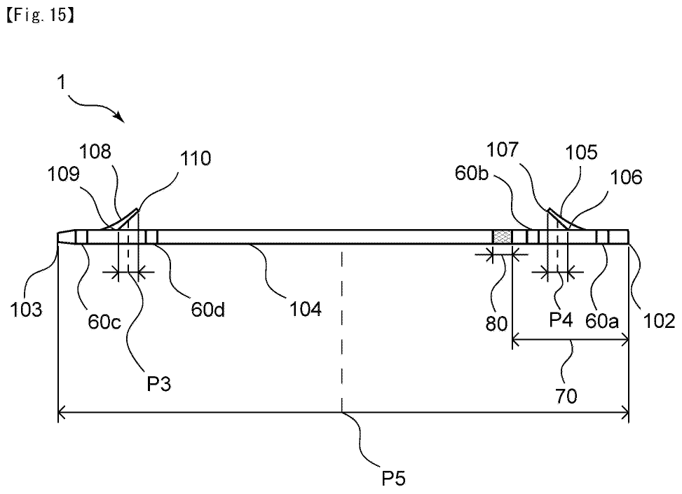

As shown in FIG. 15, preferably, the tubular member 104 includes, radially outward of the tubular member 104, a supporting member 60 on at least one of on the distal side of the midpoint P3 of the base end 109 and the free end 110 of the distal flap 108, on the proximal side of the free end 110 of the distal flap 108 and on the distal side of the midpoint P5 of the tubular member 104, on the proximal side of the midpoint P4 of the base end 106 and the free end 107 of the proximal flap 105, and on the distal side of the free end 107 of the proximal flap 105 and on the proximal side of the midpoint P5 of the tubular member 104.

Specifically, since the tubular member 104 includes the distal side first supporting member 60c on the distal side of the midpoint P3 between the base end 109 and the free end 110 of the distal flap 108, when stress is applied to the base end 109 of the distal flap 108, it is possible to prevent the base end 109 from being torn and the distal flap 108 from being broken.

The strength of the portion provided with the distal side second supporting member 60d of the tubular member 104 can be increased by including the distal side second supporting member 60d on the proximal side of the free end 110 of the distal flap 108 and on the distal side of the midpoint P5 of the tubular member 104. As a result, pushability in the vicinity of the distal flap 108 of the in-vivo indwelling tube 1 can be improved.

Since the proximal side first supporting member 60a is arranged on the proximal side of the midpoint P4 between the base end 106 and the free end 107 of the proximal flap 105, when stress is applied to the base end 106 of the proximal flap 105, it is possible to prevent the base end 106 from being torn and the proximal flap 105 from being broken.

The strength of the portion provided with the proximal side second supporting member 60b of the tubular member 104 can be increased by including the proximal side second supporting member 60b on the distal side of the free end 107 of the proximal flap 105 and on the proximal side of the midpoint P5 of the tubular member 104. As a result, pushability in the vicinity of the proximal flap 105 of the in-vivo indwelling tube 1 can be improved.

As shown in FIG. 15, the tubular member 104 includes a first region 70 and a second region 80 sequentially from the proximal side of the tubular member 104, and the colors of the first region 70 and the second region 80 are preferably different from each other on the distal side of the base end 106 of the proximal flap 105. When the colors of the first region 70 and the second region 80 are different from each other, this means that at least one of the hue, the lightness, and the saturation defined in JIS Z8721 is different between the color of the first region 70 and the color of the second region 80. Since the tubular member 104 includes the first region 70 and the second region 80, and the colors of the first region 70 and the second region 80 are different from each other, the position of the proximal flap 105 of the in-vivo indwelling tube 1 can be easily confirmed with an endoscope when transporting the in-vivo indwelling tube 1 to a desired location in the lumen in the living body. If the color of the second region 80 is a color different from that of the first region 70 and is a color that is easily visible under an endoscope, the boundary between the first region 70 and the second region 80 can be easily confirmed and the proximal flap 105 of the in-vivo indwelling tube 1 can be easily confirmed. For example, the color of the first region 70 may be a low lightness color such as black and the color of the second region 80 may be a high lightness color such as yellow, or the color of the first region 70 may be a high lightness color and the color of the second region 80 may be a low lightness color.

Apart from the first region 70 and the second region 80, the tubular member 104 may or may not include a region different in color from at least either the first region 70 or the second region 80 on the proximal side of the base end 106 of the proximal flap 105. If the tubular member 104 includes a region different in color on the proximal side of the base end 106 of the proximal flap 105, the proximal end 102 and the distal end 103 of the in-vivo indwelling tube 1 can be easily distinguished. If the tubular member 104 does not include a region different in color on the proximal side of the base end 106 of the proximal flap 105, the first region 70 becomes noticeable and the visibility of the first region 70 in the endoscope is enhanced.

A method for making the colors of the first region 70 and the second region 80 different from each other includes, for example, a method of coloring at least one of the portion to become the first region 70 and the portion to become the second region 80 in the tubular member 104, a method of disposing a film or a tubular member different in color from the tubular member 104 in at least one of the portion to become the first region 70 and the portion to become the second region 80, and the like. The method of coloring includes a method of applying paint, a method of dyeing with dye, and the like. Among them, it is preferable to apply and color a paint having a color different from that of the tubular member 104 to the portion to become the first region 70 of the tubular member 104. The visibility of the position of the proximal flap 105 of the in-vivo indwelling tube 1 in the endoscope can be enhanced by making the colors of the first region 70 and the second region 80 different from each other as described above. Furthermore, the tubular member 104 may include a region of a color different from the second region 80 on the distal side of the second region 80.

The first region 70 may be configured by the supporting member 60. Specifically, for example, the color of the proximal side second supporting member 60b provided on the distal side of the free end 107 of the proximal flap 105 of the tubular member 104 and on the proximal side of the midpoint P5 of the tubular member 104 may be a color different from the color of the first region 70, and the proximal side second supporting member 60b may be provided on the distal side of the first region 70 and may be the second region 80. The second region 80 may be a region including the proximal side second supporting member 60b.

The length in an axial direction of the first region 70 and the second region 80 can be appropriately set so as to be easily visible. At least the first region 70 may be a portion that starts from the proximal side of the base end 106 of the proximal flap 105 of the tubular member 104 and ends on the distal side of the base end 106 of the proximal flap 105. The proximal end of the first region 70 may coincide with the proximal end 102 of the tubular member 104.

The proximal end of the first region 70 is preferably arranged on the distal side of the position P2 of the tubular member 104 corresponding to the free end 107 of the proximal flap 105. The position P2 of the tubular member 104 corresponding to the free end 107 of the proximal flap 105 is the position where the free end 107 of the proximal flap 105 comes into contact with the tubular member 104 when the proximal flap 105 is arranged along the tubular member 104 and the proximal flap 105 is closed.

When the tubular member 104 includes the larger diameter portion 111, the distal side second supporting member 60d, the first region 70, and the second region 80, the maximum outer diameter of the larger diameter portion 111 is preferably larger than the average outer diameter of the tubular member 104 between the position P1 of the tubular member 104 corresponding to the free end 110 of the distal flap 108 or the proximal end of the distal side second supporting member 60d and the position P2 of the tubular member 104 corresponding to the free end 107 of the proximal flap 105 or the distal end of the first region 70.

When the tubular member 104 includes the smaller diameter portion 114, the distal side second supporting member 60d, the first region 70, and the second region 80, the minimum outer diameter of the smaller diameter portion 114 is preferably smaller than the average outer diameter of the tubular member 104 between the position P1 of the tubular member 104 corresponding to the free end 110 of the distal flap 108 or the proximal end of the distal side second supporting member 60d and the position P2 of the tubular member 104 corresponding to the free end 107 of the proximal flap 105 or the distal end of the first region 70.

As described above, a method for producing an in-vivo indwelling tube having one end and the other end includes a first process of arranging, in a lumen of the first tubular member, the other end of a second tubular member, a second process of joining the first tubular member and the second tubular member, a third process of arranging the second tubular member in a lumen of a third tubular member that its length in an axial direction is shorter than the second tubular member, a fourth process of joining the second tubular member and the third tubular member, a fifth process of arranging one end of the second tubular member in a lumen on the other end side of the fourth tubular member including a flap having a base end and a free end, and a sixth process of joining the second tubular member and the fourth tubular member. The in-vivo indwelling tube that is itself flexible but has increased flap strength can be obtained by producing the in-vivo indwelling tube through such a production method.

The present application claims the benefit of priority based on Japanese patent application number 2017-115569 filed on Jun. 13, 2017. The entire content of the specification of Japanese patent application number 2017-115569 filed on Jun. 13, 2017 is incorporated herein by reference.

### DESCRIPTION OF REFERENCE SIGNS

1: in-vivo indwelling tube

2: delivery system

3: inner catheter

4: outer catheter

5: suture thread

6: insertion auxiliary tube

10: first tubular member

11: proximal end of first tubular member

20: second tubular member

21: proximal end of second tubular member

22: distal end of second tubular member

30: third tubular member

31: proximal end of third tubular member

32: distal end of third tubular member

40: fourth tubular member

41: proximal end of fourth tubular member

42: distal end of fourth tubular member

43: flap

44: base end of flap

45: free end of flap

50: core material

60: supporting member

60a: proximal side first supporting member

60b: proximal side second supporting member

60c: distal side first supporting member

60d: distal side second supporting member

70: first region

80: second region

102: proximal end of in-vivo indwelling tube

103: distal end of in-vivo indwelling tube

104: tubular member

105: proximal flap

106: base end of proximal flap

107: free end of proximal flap

108: distal flap

109: base end of distal flap

110: free end of distal flap

111: larger diameter portion

112: proximal end of larger diameter portion

113: distal end of larger diameter portion

114: smaller diameter portion

115: proximal end of smaller diameter portion

116: distal end of smaller diameter portion

P1: position of tubular member corresponding to free end of distal flap

P2: position of tubular member corresponding to free end of proximal flap

P3: midpoint between base end and free end of distal flap

P4: midpoint between base end and free end of proximal flap

P5: midpoint of tubular member

201: conventional in-vivo indwelling tube

202: proximal end of conventional in-vivo indwelling tube

203: distal end of conventional in-vivo indwelling tube

204: conventional tubular member

205: conventional proximal flap

206: base end of conventional proximal flap

207: free end of conventional proximal flap

208: conventional distal flap

209: base end of conventional distal flap

210: free end of conventional distal flap

214: conventional smaller diameter portion

215: proximal end of conventional smaller diameter portion

216: distal end of conventional smaller diameter portion

1. A method for producing an in-vivo indwelling tube having one end and the other end comprising the following steps:
(1) arranging the other an end of a second tubular member in a lumen of the first tubular member;
(2) joining the first tubular member and the second tubular member;
(3) arranging the second tubular member in a lumen of a third tubular member so that its length in an axial direction is shorter than the second tubular member;
(4) joining the second tubular member and the third tubular member;
(5) arranging another end of the second tubular member in a lumen on the other end side of a fourth tubular member including a flap having a base end and a free end; and
(6) joining the second tubular member and the fourth tubular member.

2. The method for producing the in-vivo indwelling tube according to claim 1, wherein
a thickness of the second tubular member is thinner than thicknesses of the first tubular member, the third tubular member, and the fourth tubular member.

3. The method for producing the in-vivo indwelling tube according to claim 1, wherein
a thickness of the flap of the fourth tubular member is thicker than the thickness of at least either the first tubular member or the third tubular member.

4. The method for producing the in-vivo indwelling tube according to claim 1, wherein
a thickness of the flap of the fourth tubular member is thinner than the thickness of at least either the first tubular member or the third tubular member.

5. The method for producing the in-vivo indwelling tube according to claim 1, wherein
a type A durometer hardness of a material that constitutes the fourth tubular member is higher than a type A durometer hardness of a material that constitutes the first tubular member, the second tubular member, and the third tubular member.

6. The method for producing the in-vivo indwelling tube according to claim 1, wherein
an inner diameter of one end of the first tubular member is larger than an inner diameter of the other end of the first tubular member.

7. The method for producing the in-vivo indwelling tube according to claim 1, wherein
step (4), the first tubular member and the third tubular member are joined.

8. The method for producing the in-vivo indwelling tube according to claim 1, wherein
(6), the third tubular member and the fourth tubular member are joined.

9. The method for producing the in-vivo indwelling tube according to claim 1, wherein
before step (6), one end of the third tubular member is arranged on one end side or the other end side of the fourth tubular member of the base portion of the flap of the fourth tubular member.

10. The method for producing the in-vivo indwelling tube according to claim 1, wherein
steps (2) and (4), or steps (2), (4) and (6) are performed by a simultaneous heating process.

11. The method for producing the in-vivo indwelling tube according to claim 1, further comprising, before step (1), a step of arranging a core material in a lumen of the second tubular member.

12. The method for producing the in-vivo indwelling tube according to claim 1, further comprising, after step (6), a step of arranging at least one supporting member on an outer surface of the tube at either one end side of the base end of the flap of the fourth tubular member, or the other end side of the base end of the flap of the fourth tubular member and one end side of a midpoint of the first tubular member.

13. An in-vivo indwelling tube comprising:
a tubular member having a proximal side and a distal side;
a proximal flap, having a base end on a proximal side and a free end on a distal side, on the proximal side of the tubular member; and
a distal flap, having a base end on a distal side and a free end on a proximal side, on the distal side of the tubular member, wherein
the tubular member has a larger diameter portion having a maximum outer diameter larger than an average outer diameter of the tubular member between a position of the tubular member corresponding to the free end of the distal flap and a position of the tubular member corresponding to the free end of the proximal flap, and
the larger diameter portion is on at least either the proximal side of the base end of the proximal flap or the distal side of the base end of the distal flap.

14. An in-vivo indwelling tube comprising:
a tubular member having a proximal side and a distal side;
a proximal flap, having a base end on a proximal side and a free end on a distal side, on the proximal side of the tubular member; and
a distal flap, having a base end on a distal side and a free end on a proximal side, on the distal side of the tubular member, wherein
the tubular member has a smaller diameter portion having a minimum outer diameter smaller than an average outer diameter of the tubular member between a position of the tubular member corresponding to the free end of the distal flap and a position of the tubular member corresponding to the free end of the proximal flap, and
the smaller diameter portion is on at least either between the base end of the proximal flap and the position on the proximal side of the free end of the proximal flap when the proximal flap is in a closed state or between the base end of the distal flap and a position on the distal side of the free end of the distal flap when the distal flap is in a closed state.

15. The in-vivo indwelling tube according to claim 14, wherein
the tubular member has a hole in the smaller diameter portion.

16. The in-vivo indwelling tube according to claim 13, further comprising at least one supporting member on an outer surface of the tubular member, wherein
the at least one supporting member is disposed on at least one of
the distal side of a midpoint of the base end and the free end of the distal flap,
a proximal side of the free end of the distal flap and the distal side of the midpoint of the tubular member,
a proximal side of a midpoint of the base end and the free end of the proximal flap, and
a distal side of the free end of the proximal flap and the proximal side of the midpoint of the tubular member.

17. The in-vivo indwelling tube according to claim 13, wherein
the tubular member includes a first region and a second region sequentially from the proximal side of the tubular member, and
colors of the first region and the second region differ from each other on the distal side of the base end of the proximal flap.

18. The in-vivo indwelling tube according to claim 13, wherein
a thickness of the distal flap or the proximal flap is thinner than a thickness of a proximal end of the tubular member.

19. The in-vivo indwelling tube according to claim 13, wherein
the thickness of the proximal flap is an average thickness or more of the tubular member between the position of the tubular member corresponding to the free end of the proximal flap and the position of the tubular member corresponding to the free end of the distal flap.

20. The in-vivo indwelling tube according to claim 13, wherein
a type A durometer hardness of a material that constitutes the proximal flap or the distal flap is higher than an average type A durometer hardness of a material that constitutes the tubular member between the position of the tubular member corresponding to the free end of the distal flap and the position of the tubular member corresponding to the free end of the proximal flap.

21. The in-vivo indwelling tube according to claim 14, further comprising at least one supporting member on an outer surface of the tubular member, wherein
the at least one supporting member is disposed on at least one of
the distal side of a midpoint of the base end and the free end of the distal flap,
a proximal side of the free end of the distal flap and the distal side of the midpoint of the tubular member,
a proximal side of a midpoint of the base end and the free end of the proximal flap, and
a distal side of the free end of the proximal flap and the proximal side of the midpoint of the tubular member.

22. The in-vivo indwelling tube according to claim 14, wherein
the tubular member includes a first region and a second region sequentially from the proximal side of the tubular member, and
colors of the first region and the second region differ from each other on the distal side of the base end of the proximal flap.

23. The in-vivo indwelling tube according to claim 14, wherein
a thickness of the distal flap or the proximal flap is thinner than a thickness of a proximal end of the tubular member.

24. The in-vivo indwelling tube according to claim 14, wherein
the thickness of the proximal flap is an average thickness or more of the tubular member between the position of the tubular member corresponding to the free end of the proximal flap and the position of the tubular member corresponding to the free end of the distal flap.

25. The in-vivo indwelling tube according to claim 14, wherein
a type A durometer hardness of a material that constitutes the proximal flap or the distal flap is higher than an average type A durometer hardness of a material that constitutes the tubular member between the position of the tubular member corresponding to the free end of the distal flap and the position of the tubular member corresponding to the free end of the proximal flap.
